(19) United States
(12) Patent Application Publication
(10) Pub. No.: US 2010/0075393 A1
(43) Pub. Date: Mar. 25, 2010

(54) Aqueous microfabrication of functional bioelectronic architectures

(75) Inventors: Jason B. Shear, Austin, TX (US); Keith J. Stevenson, Austin, TX (US); Ryan T. Hill, Austin, TX (US); Jennifer L. Lyon, Austin, TX (US)

Correspondence Address: CHALKER FLORES, LLP, 2711 LBJ FRWY, Suite 1036, DALLAS, TX, 75234, US

(73) Assignee: Board of Regents,The University of Texas System, Austin, TX (US)

(21) Appl. No.: 11/439,552

(22) Filed: May 23, 2006

Related U.S. Application Data
(60) Provisional application No. 60/683,883, filed on May 23, 2005.

(51) Int. Cl. C12N 9/08; C07K 1/00; C08J 3/28; C12N 9/96; C07H 1/00; B01J 19/00; C07K 14/00; C07K 2/00; C07H 21/00; C07K 14/80; C12N 9/02; C07K 14/75; C07K 14/76; C07K 14/805

(52) U.S. Cl. 435/192; 530/402; 522/104; 435/188; 522/157; 522/155; 522/153; 522/152; 522/166; 536/25.3; 536/124; 422/188; 530/350; 530/300; 536/23.1; 536/1.11; 530/401; 435/189; 530/382; 530/395; 530/363; 530/400

(57) ABSTRACT

The present invention is an apparatus, system and method for forming nanoscale architectures having nanoparticles bound thereto. The present invention provides a photon beam crosslinked polymer matrix, wherein the crosslinked matrix includes one or more polymers crosslinked to one or more crosslinking agents and one or more protein-coated metal nanoparticles.

### CROSS-REFERENCE TO RELATED APPLICATIONS

This application claims priority to U.S. Provisional Application Ser. No. 60/683,883, filed May 23, 2005.

The U.S. Government may own certain rights to this invention under National Science Foundation Grant No. 0317032 and 0134884.

### TECHNICAL FIELD OF THE INVENTION

The present invention relates generally to a system, method and apparatus for forming nanoscale architectures, and in particular, to multi-photon excitation crosslinking and metallization of polymers for the fabrication of architecture on the nanometer-scale.

### BACKGROUND OF THE INVENTION

Without limiting the scope of the invention, its background is described in connection with a nanometer-scale architectures fabrication system, method, and apparatus, as an example.

The construction of synthetic nanocomposites and materials with nanometer-scale domains has received considerable attention with the advancements of material science, chemistry, biology and engineering. Architectures and complex structures on the nanometer-scale are common in biological systems and largely responsible for many of there properties. Generally, nanoscale structures have dimensions or features in the range of about 2 to about 100 nm (e.g., a nanometer is 1 nm or 10 angstroms) which is on the size range of macromolecules such as DNA, RNA, PNA, proteins and protein complexes. Such nanocomposites are expected to possess unique properties similar to their biological counterparts as a result of their sophisticated nanoarchitectures.

For the most part, conventional processing techniques have been unable to achieve nanometer-scale architectural with the nanoscale control of the fabrication. Thus, one of the goals has been the development of methods for constructing synthetic composites with a degree of nanometer-scale organization similar to that in biological systems while retaining the ability to incorporate modern engineering materials. For example, elongated ceramic particles have been precipitated within polymer matrices by drawing the polymer during the precipitation reaction (see Burdon, J.; Calvert, P. In Hierachially Structured Materials); CdS (see, Nelson, et al., Mater. Sci. Eng. C2:133 (1995)) has been precipitated in liquid-crystalline polymers; metals have been electrodeposited inside the pores of commercial nanopore membranes (see, Martin, Chem. Mater. 8:1739 (1996)); and polymers have been grown within the cavities of layered inorganic structures (see, Okada, et al., Mater. Sci. Eng. C C3(2):109 (1995)) and zeolites (see, Frisch, et al., Chem. Mater. 8:1735 (1996)). However, none of these methods allow control over both nanometer-scale architecture and composition.

Other conventional processing techniques have been unable to achieve nanometer-scale architectural entirely and/or unable to adequately control the fabrication on the nanoscale range. One method currently used to make two-dimensional structures is photolithography (e.g., X-ray and deep UV). However, one limitation to photolithography is the lack of fine control and the inability to make complex or curved architectures. Furthermore, the technique limits the movement in the z-direction; and thus, does not allow complex, curved three-dimensional surfaces. Three-dimensional objects produced by photolithographic methods have therefore been essentially limited to columnar structures larger than 150 nm.

A technique for generating three-dimensional microscale objects is described by S. Maruo, O. Nakamura, and S. Kawata et al. in “Three Dimensional Microfabrication With Two-Photon-Absorbed Photopolymerization”, Optics Letters, Vol. 22, No. 2, pp. 132-134 (1997), which is incorporated herein by reference in its entirety. Maruo et al. discloses microscale structures formed by subjecting urethane acrylate monomers and oligomers to near-infrared laser light in a non-solvent system. However, the structures disclosed are not on the nanoscale and only synthesis in a non-solvent system is described and thus not applicable to biomolecules.

The foregoing problems have been recognized for many years and while numerous solutions have been proposed, none of them adequately address all of the problems in a single device, e.g., nanoscale size, fine control and complex nanoscale architecture, while providing ordered nanocomposites, architectures with complex structures on the nanometer-scale that are well-defined and tuneable to allowing nanoscale control of the fabrication, architecture and composition.

### SUMMARY OF THE INVENTION

The inventors recognized that future microelectronic components and devices require ultra-small sensing and on-chip power generation applications. Therefore, requiring lithographic methods that can fabricate higher surface area, 3D bioelectronic architectures, unlike the fabrication methods current used that are inherently 2D techniques that have not proven useful for creating complex 3D assemblies and involve expensive masks, complicated stamping, chemical etching or methods that are combinations of both, e.g., conventional photolithography and microcontact printing.

The present invention use a direct-write lithography that relies on non-linear multiphoton excitation (MPE) to spatially confine polymerization and crosslinking reactions to volumes as small as about 1 fL (1 μm³) (21, 22). For example, a femtosecond pulsed laser is directed into an inverted microscope containing a high numerical aperture (NA) objective, and photocrosslinked structures are directly “written” by using an x-y stage and/or galvanometer-controlled mirrors to translate the laser beam focus through a solution containing protein and a photosensitizer or cross linking agent. Nonlinear excitation of the photosensitizer (e.g., flavin adenine dinucleotide, methylene blue) promotes covalent bond formation between protein residue side-chains, a process that creates a dense matrix of entangled macromolecules that often retains native functionality of the protein building blocks.

Another example of the present invention includes a redox-active photocrosslinked protein features at write speeds as fast as about 10³ μm²/sec with 250-nm resolution on a variety of substrates, including silica, ITO, and gold. Photocrosslinked avidin retains a high affinity for biotin (and biotinylated ligands) and electrochemical studies indicate that immobilized cytochrome c matrices that consist of several hundred monolayers may remain redox-active even after extended electrochemical conditioning.

The present invention allows for fabrication of a robust biomaterial composites highly resistant to structural failure even when sonicated extensively in harsh detergents or surfactants. The photocrosslinked protein matrices can serve as efficient scaffolds for creating bio-metallic conduits, a capability that will be of substantial value in fabricating conductive interconnects and electronic circuitry for wiring bioelectrode components.

In the present invention, metal nanoparticle delivery is targeted to specific protein matrices using protein-protein interactions. In one embodiment, gold nanoparticles are coated with a protein that has an isoelectric point (pI) significantly different from that of the matrix protein; by incubating nanoparticles and crosslinked structures in a medium buffered at a pH intermediate to the two pIs, high densities of gold (e.g., about 1 particle per 2500 nm²) can be bound from solution. After binding, metal nanoparticles (e.g., initially, about 5 nm) can be grown using electroless deposition procedures to create essentially continuously metallized materials.

The multiphoton photodeposition approach of the present invention provides biopolymers as scaffolds for electronic and electrochemical materials by supporting protein matrices can be fabricated with well-defined morphologies in three dimensions and with minimum feature sizes that approach those reported for randomly placed biopolymer-templated wires. The present invention also includes a direct-write instrument that enables high-resolution fabrication and characterization of mathematically defined matrices with arbitrary, three-dimensional morphologies. The present invention includes a closed-loop piezo electric stage with about ±1 nm lateral positioning and about ±5 nm repositioning accuracy, an inverted microscope interfaced with an ultrafast laser for multiphoton excitation, several detectors for materials characterization (e.g., spectroscopy, microscopy, and electrochemistry), and lithography software to drive the stage in arbitrary directions while controlling an optical shutter to limit sample exposure. The integrated approach avoids problems inherent to transport of samples between instruments and will facilitate optimization of the fabrication process (e.g., crosslinking densities, structural characteristics, contact resistances, bioactivities).

In accordance with the present invention, a method and apparatus are provided that metallized biomolecular nanostructure of crosslinked polymers bound by protein coated metal nanoparticles. The metallized biomolecular scaffold nanostructure may be in integral contact with a support surface, extends as freestanding structures through a solution or have regions in contact with a support surface and other regions that extend as freestanding structures. The metallized biomolecular scaffold may be bound with nanoparticles made from one or more pure metals, one or more semiconductor, one or more metal oxides and combinations and mixtures thereof. Therefore, the properties of the metallized biomolecular scaffold structure may be influenced by the nanoparticles bound thereto. For example, gold nanoparticles bound to the metallized biomolecular scaffold structure will allow the conduction of electrons or electricity.

The present invention provides various methods of making metallized micro- and nano-architectures. For example, one method of making a metallized biomolecular scaffold includes crosslinking a polymer matrix with a photon beam to form a crosslinked matrix. The crosslinked matrix includes one or more polymers crosslinked to one or more crosslinking agents and binding one or more metal protein coated nanoparticles with the crosslinked matrix.

In another example, the present invention provides a system for forming a nanoscale structure in a solution that includes a chamber suitable for nanoparticle metallization and positioned to receive one or more photons from an optical system comprising an imaging mechanism interfaced with a multiphoton excitation laser that crosslinks one or more polymers and one or more photosensitizers prior to binding of one or more metal nanoparticles.

The present invention also provides an electrical conductive nanoscale architectural matrix having one or more metal nanoparticles bound to an architectural matrix comprising a multi-photon beam induced crosslink between one or more polymers and one or more photosensitizers.

### BRIEF DESCRIPTION OF THE DRAWINGS

For a more complete understanding of the features and advantages of the present invention, reference is now made to the detailed description of the invention along with the accompanying figures and in which:

FIGURES 1a-1c are scanning electron micrograph images of high-density metallization of matrices comprised of photocrosslinked cytochrome c;

FIGS. 2a-2c are transmission images illustrating detailed control of metallized-protein architectures in two and three dimensions;

FIG. 3a is a graph of the conductivity measurements of metallized cytochrome c matrices;

FIG. 3b SEM depicting the metallized cytochrome c matrix after severing with a focused ion beam (FIB); and

FIGS. 4a and 4b are high-density metallization of matrices comprised of photocrosslinked cytochrome c.

### DETAILED DESCRIPTION OF THE INVENTION

While the making and using of various embodiments of the present invention are discussed in detail below, it should be appreciated that the present invention provides many applicable inventive concepts that can be embodied in a wide variety of specific contexts. The terminology used and specific embodiments discussed herein are merely illustrative of specific ways to make and use the invention and do not delimit the scope of the invention.

In accordance with the present invention, a method and apparatus are provided that metallized biomolecular nanostructure of crosslinked polymers bound by metal nanoparticles. The metallized biomolecular scaffold nanostructure may be in integral contact with a support surface, extends as freestanding structures through a solution or have regions in contact with a support surface and other regions that extend as freestanding structures. The metallized biomolecular scaffold may be bound with nanoparticles made from one or more pure metals, one or more semiconductor, one or more metal oxides and combinations and mixtures thereof. Furthermore, persons of ordinary skill in the art will recognize that a variety of proteins may be used to coat the metallized nanoparticle. Therefore, the properties of the metallized biomolecular scaffold structure may be influenced by the nanoparticles bound thereto. For example, gold Nanoparticles bound to the metallized biomolecular scaffold structure will allow the conduction of electrons or electricity.

The present invention provides various method of making metallized micro- and nano-architectures including crosslinking a polymer matrix with a photon beam to form a crosslinked matrix. The crosslinked matrix includes one or more polymers crosslinked to one or more crosslinking agents. Furthermore, the crosslinked matrix binds one or more metal nanoparticles which are coated with one or more proteins.

In some instances the photon beam from a laser is used to form a crosslinked matrix; however in some instances more than one laser may be used to crosslink the matrix. The skilled artisan will recognize that a variety of lasers may be used including a Ti:O₂ laser and a Nd:YAG laser. In addition the emission may be from a wavelength in at least one of the deep red, red, infrared, visible and ultraviolet segments of the electromagnetic spectrum. In some embodiments, the photon beam is scanned, while in other embodiments a stage mechanism is moved. Such movable stages are known in the art, (e.g., an x-y stage, a closed-loop piezo electric stage a galvanometer-controlled mirrors to translate the laser beam focus through a solution containing a protein and a photosensitizer.

The polymers and the crosslinkers (e.g., photosensitizer) may be biological polymers, synthetic polymers or combinations thereof. Furthermore, the polymers may be heteropolymers or homopolymers and the crosslinkers may be of similar of different structure as well. For example polymers may include cytochrome c, cytochrome c oxidase, cytochrome c peroxidase, horseradish peroxidase, fibrinogen, trimethylolpropane triacrylate, avidin, bovine serum albumin, and the heme proteins, myoglobin or combinations and mixtures thereof. Furthermore, the polymer may be made of monomers of the same composition or different compositions including one or more photopolymerizable organic monomers, photopolymerizable inorganic monomers, cross-linkers, monomers having at least one olefinic bond, oligomers having at least one olefinic bond, polymers having at least one olefinic bond, olefins, halogenated olefins, acrylates, methacrylates, acrylamides, bisacrylamides, styrenes, epoxides, cyclohexeneoxide, amino acids, peptides, proteins, fatty acids, lipids, nucleotides, oligonucleotides, synthetic nucleotide analogues, nucleic acids, sugars, carbohydrates, cytokines and combinations or mixtures thereof. Additionally the monomers and/or the polymers may be functionalized with chemical or biological components (e.g., biotin). Crosslinkers may include flavin adenine dinucleotide, heme proteins, cytochrome c, methylene blue or combinations and mixtures thereof. Additionally, the nanoparticles may include one or more pure metals (e.g., gold, silver, copper, etc.), metal alloys, one or more semiconductor, one or more metal oxides and combinations and mixtures thereof. The nanoparticles may be of different sizes and may be monodisperse or polydisperse depending on the particular application.

In another example, the present invention provides a system for forming a nanoscale structure in a solution including a chamber suitable for nanoparticle metallization and positioned to receive one or more photons from an optical system comprising an imaging mechanism interfaced with a multiphoton excitation laser that crosslinks one or more polymers and one or more photosensitizers prior to binding of one or more metal nanoparticles.

In some instances, the photons used to form a crosslinked matrix is a laser; however, more than one laser may be used for crosslinking. The skilled artisan will recognize that a variety of lasers may be used including a Ti:O₂ laser and a Nd:YAG laser. In addition the emission may be from a wavelength in at least one of the deep red, red, infrared, visible and ultraviolet segments of the electromagnetic spectrum. In some embodiments, the photon beam is scanned, while in other embodiments a stage mechanism is moved to position the chamber. Such movable stages are known in the art, (e.g., an x-y stage, a closed-loop piezo electric stage). A galvanometer-controlled mirror may also be used to translate the laser beam focus through a solution containing protein and a photosensitizer or crosslinker.

The polymers and the crosslinkers may be biological polymers, or synthetic polymers or combinations thereof. Furthermore, the polymers may be heteropolymers or homopolymers and the crosslinkers may be of similar of different structure as well. For example, polymers may include cytochrome c, cytochrome c oxidase, cytochrome c peroxidase, horseradish peroxidase, fibrinogen, trimethylolpropane triacrylate, avidin, bovine serum albumin, and the heme proteins, myoglobin or combinations and mixtures thereof. Furthermore, the polymer may be made of monomers of the same composition or different compositions including one or more photopolymerizable organic monomers, photopolymerizable inorganic monomers, cross-linkers, monomers having at least one olefinic bond, oligomers having at least one olefinic bond, polymers having at least one olefinic bond, olefins, halogenated olefins, acrylates, methacrylates, acrylamides, bisacrylamides, styrenes, epoxides, cyclohexeneoxide, amino acids, peptides, proteins, fatty acids, lipids, nucleotides, oligonucleotides, synthetic nucleotide analogues, nucleic acids, sugars, carbohydrates, cytokines and combinations or mixtures thereof. Additionally the monomers and/or the polymers may be functionalized with chemical or biological components (e.g., biotin). Crosslinkers may include flavin adenine dinucleotide, heme proteins, cytochrome c, methylene blue or combinations and mixtures thereof.

The nanoparticles may include one or more pure metals (e.g., gold, silver, copper, etc.) one or more semiconductors, one or more metal oxides and combinations and mixtures thereof. The nanoparticles may be of different sizes and may be monodisperse or polydisperse depending on the particular application. The skilled artisan will recognize the various methods to make nanoparticles and the vast array of nanoparticle compositions and variety of proteins that may be used to coat the nanoparticle. The present system may also include one or more detectors to record spectroscopic characteristics, optical characteristics, electrochemistry characteristics or a combination thereof.

In addition, the present invention provides an electrical conductive nanoscale architectural matrix including one or more metal nanoparticles bound to an architectural matrix comprising a multi-photon beam induced crosslink between one or more polymers and one or more photosensitizers. In some instances, the multi-photon beam used to form a crosslinked matrix is a laser; however more than one laser may be used to crosslink the matrix. The skilled artisan will recognize that a variety of lasers may be used including a Ti:O₂ laser and a Nd:YAG laser. In addition the emission of the multi-photon may be from a wavelength in at least one of the deep red, red, infrared, visible and ultraviolet segments of the electromagnetic spectrum. In some embodiments, the multi-photon beam is scanned, while in other embodiments a stage mechanism is moved. Such movable stages are known in the art, e.g., an x-y stage, a closed-loop piezo electric stage. In addition a galvanometer-controlled mirrors may be used to translate the laser beam focus through a solution containing protein and a photosensitizer or crosslinker.

The polymers and the photosensitizers (e.g., crosslinker) may be biological polymers, or synthetic polymers or combinations thereof. Furthermore, the polymers may be heteropolymers or homopolymers and the photosensitizers may be of similar of different structure as well. For example, polymers may include cytochrome c, cytochrome c oxidase, cytochrome c peroxidase, horseradish peroxidase, fibrinogen, trimethylolpropane triacrylate, avidin, bovine serum albumin, and the heme proteins, myoglobin or combinations and mixtures thereof. Furthermore, the polymer may be made of monomers of the same composition or different compositions including one or more photopolymerizable organic monomers, photopolymerizable inorganic monomers, cross-linkers, monomers having at least one olefinic bond, oligomers having at least one olefinic bond, polymers having at least one olefinic bond, olefins, halogenated olefins, acrylates, methacrylates, acrylamides, bisacrylamides, styrenes, epoxides, cyclohexeneoxide, amino acids, peptides, proteins, fatty acids, lipids, nucleotides, oligonucleotides, synthetic nucleotide analogues, nucleic acids, sugars, carbohydrates, cytokines and combinations or mixtures thereof. Additionally the monomers and/or the polymers may be functionalized with chemical or biological components (e.g., biotin). photosensitizers may include flavin adenine dinucleotide, heme proteins, cytochrome c, methylene blue or combinations and mixtures thereof.

The electrical conductive nanoscale architectural matrix of the present invention may include one or more protein coated metal nanoparticles bound to an architectural matrix having a multi-photon beam induced crosslink between one or more polymers and one or more photosensitizers. Furthermore, the device nanoscale architectural matrix may be in integral contact with a support surface and or may extend as freestanding structures through a solution or a combination thereof.

The ability of biological macromolecules to direct seeding, growth, and organization of inorganic materials offers valuable opportunities for materials synthesis. Studies of natural biomineralization processes have inspired efforts to specify the structure of inorganic materials over many length scales, from quantum dots with well-defined crystallinity to large single crystals of calcium carbonate (1, 2). Recently, several strategies have been explored for using macromolecules to scaffold electronically conductive metallic components within aqueous solutions, a goal that could provide routes for fashioning new electrochemical architectures, nanoelectronic components, and cellular interfaces. In these approaches, surface-adhered biofilaments (e.g., DNA (3-5) and polyproteins such as amyloid fibers (6), peptide nanotubes (7), and F-actin (8)) have been used as templates to grow metallic “bio-wires” through the catalytic reduction of copper, gold, and silver ions. Metallization has been initiated both directly from electrostatically associated ions or by covalently bound metalnanoparticle seeds. Although such procedures have yielded wires with radial dimensions as small as about 0.1 μm and having conductivities of about 104Ω⁻¹ cm ¹, the arrangement of such materials into functional electronic patterns faces severe challenges. In general, long biofilaments have been applied to planar substrates only with random orientation. The present invention relates to the construction of both surface-adherent and free-standing biomolecular scaffoldings for electronic components with submicron, three-dimensional control.

The present invention includes proteins that are photocrosslinked into controllably placed matrices that display high-binding capacities for functionalized metal nanoparticles; decoration of protein structures with nanoparticle-seeds followed by reductive metallization yields hybrid materials that are highly conductive. The present invention also includes building protein-based structures using a direct-write process based on scanning multiphoton excitation, matrices fabricated with feature sizes that range from hundreds of nanometers to more than a millimeter and that may either remain in integral contact with a support surface or extend into free solution, e.g., hundredth of microns to hundreds of microns. The present invention may be used to pattern a broad range of functional materials in well-defined topographies, offering new opportunities to construct advanced bioelectronic architectures.

Reagents and Materials. Bovine heart cytochrome c (cytochrome c; Sigma-Aldrich, St. Louis, Mo., C3131), avidin (Molecular Probes, Eugene, Oreg., A-887) and flavin adenine dinucleotide (FAD; Sigma-Aldrich, F-6625) were stored desiccated at about −20° C. Horse skeletal muscle myoglobin (Sigma-Aldrich, M-0630), bovine serum albumin (BSA; Equitech-Bio, Kerrville, Tex., BAH64-0100), and methylene blue (Sigma-Aldrich, M-4159) were stored undesiccated at about −20° C., about 4° C., and about room temperature, respectively. A concentrated stock solution of fluorescein-biotin (Molecular Probes, B-1370) in DMSO was stored at about 4° C. Catalytic gold enhancement solution was purchased from Nanoprobes (Yaphank, N.Y., 2112) and solutions of gold nanoparticles (e.g., about 5 nm diameter) decorated with biotinylated BSA (b-BSA), biotinylated HRP (b-HRP) and unmodified HRP were purchased from EY Labs (San Mateo, Calif., GB-01, GB-02, GP-03); each solution was stored at about 4° C. All reagents were used as received. H₂O was purified using a Barnstead NANOpure system (resistance, >18 MΩ). Glass coverslips (No. 1 thickness) were purchased from Erie Scientific (Portsmouth, N.H.).

Photolithographic modification of coverslips. Coverslips were coated with indium tin oxide (ITO; Metavac, Inc., Holtsville, N.Y.) and patterned using standard photolithographic methods, yielding non-conductive barriers of bare glass (e.g., generally, 50-100 μm) between conductive regions of ITO. ITO coverslips (e.g., coating thickness, about 100 to 200 nm) were spin-coated with 1,1,1,3,3,3-hexamethyldisilazine (HMDS, Sigma) at about 5000 rpm for about 30 seconds, followed by the positive photoresist, AZ 5214E (e.g., about 5000 rpm for about 60 seconds). Coated coverslips were prebaked at about 120° C. for about 80 seconds before being masked with an aluminum stencil (UTZ Technologies, San Marcos, Calif.) and exposed to UV radiation (e.g., about 460-Watt lamp for about 15 seconds; ABM Instruments, Santa Barbara, Calif.). After exposure, coverslips were treated immediately with about a 20% solution of developer (AZ 400K diluted in H₂O); ITO was etched in 1:3 HCl:HNO₃ solution for 120 seconds and was cleaned from coverslips by extended rinses with H₂O, acetone and isopropanol.

Multiphoton fabrication. Prior to use, patterned coverslips were subjected to three rinses with each of the following solutions: isopropanol, ethanol, and an aqueous buffer containing 18 mM phosphate and 0.1 M sodium perchlorate (pH 7.4). In most cases, surface adsorption was reduced by soaking coverslips for 10 min in phosphate/perchlorate buffer containing about 200 mg/mL BSA protein and rinsed 10 times with the buffer to be used for crosslinking. Protein matrices were generally fabricated using about 100 to about 200 mg/mL protein in about a pH 7.4 buffer solution using either FAD or methylene blue as a photosensitizer. In some examples, cytochrome c was crosslinked without addition of a separate photosensitizer. Crosslinked protein structures were written on a Zeiss Axiovert (inverted) microscope using a femtosecond titanium:sapphire (Ti:S) laser (Spectra Physics, Mountain View, Calif.) typically tuned to about 740 nm. The skilled artisan will recognize that other crosslinking sources may be used. The laser output was adjusted to approximately fill the back aperture of a high-power objective (e.g., Zeiss Fluar, 100×/1.3 numerical aperture, oil immersion); average laser powers entering the microscope were about 20 to 40 mW.

Photocrosslinked protein structures were created by raster scanning the focused laser beam within the focal plane using galvanometerdriven mirrors (BioRad MRC600 confocal scanner). In some instances, a motorized xy-stage was used to translate the position of the sample at about 3 μm/s as the beam was raster scanned, an approach capable of creating lanes of crosslinked protein that extend over distances (e.g., millimeter) ultimately limited by the stage travel. After protein crosslinking, structures were rinsed with H₂O (e.g., about 1 to about 50 times). Vertical cables (i.e., extending along the optical axis) were fabricated between opposing glass coverslips spaced (about 80 to about 100 μm) using double sided tape (3M, St. Paul, Minn.). The focus of an Olympus 40×/0.95 numerical aperture Plan Apo objective was translated from the bottom surface of the top coverslip to the top surface of the bottom coverslip through a solution containing about 400 mg/mL avidin, about 0.6 mM methylene blue, about 0.1 M NaCl, and about 20 mM HEPES (pH 7.4). Generally, additional surface-adherent protein matrix was fabricated from the positions at which a cable contacted each coverslip, thereby increasing contact area and tethering stability. Cables were washed by displacing the crosslinking solution with H₂O (e.g., four reaction volumes, about 80 μL total). In some cases, cables were subsequently labeled using 1 μM fluorescein-biotin.

Gold nanoparticle deposition and enhancement. Structures were incubated with protein-coated gold nanoparticles for about 0 to about 10 min using the about 2-mM borate buffer in which nanoparticles were supplied. Following nanoparticle exposure, matrices were rinsed with H₂O (e.g., about 30 times). In some instances, a gold enhancement solution (ca. pH 7) capable of catalytic reduction of gold onto nanoparticle seeds was applied to structures for about 3 min. Before characterization, samples were dehydrated by using five 10-min sequential washes (e.g., about 2:1 EtOH/H₂O; 2×100% EtOH; 1:1 EtOH:HMDS; 100% HMDS; all solutions vol:vol) and allowed to air dry for periods of between 20 min and several days. Patterned ITO coverslips were treated with the same protein/photosensitizer solution used for fabrication of cytochrome c matrices, but were not exposed to focused laser light. The skilled artisan will recognize that other metal coating procedures may be used. After removal of protein solution and rinsing, control coverslips were incubated with protein-coated nanoparticles and gold-enhancement solution in the same manner described for photofabrication samples.

The metal nanoparticles can be a material other than Au as well, and also need not be limited to a single material (e.g., the use of various alloy materials is contemplated). In one embodiment, the compositionally different material has a temperature coefficient of resistance that is more positive than the insulating material but less than the temperature coefficient of resistance of a metal such as Ag, Au, Cu, Pt, and AuCu. For instance, small molecules having semi-conductive properties may be metal complexes (for example, metallic hydroxyquinlates, metallic phthalocyanates, and metallic porphinates), aromatic compounds (e.g., pentance, anthracene, rubenes, pyrene, tetracene, and porphine), heteroatom containing compounds (phenyl amine, phenyl diamine, oxadiazole, trizole, carbozole, quinacridone, cyanine dyes). Additionally, semiconductor material selected from the Group of a Group III-V semiconductor, an elemental semiconductor, a Group II-VI semiconductor, a Group II-IV semiconductor, and tertiaries and quaternaries thereof may be used. Another example of the present invention includes inorganic nanowires with different compositions, e.g., Si, Ge, GaAs, CdS, CdSe, GaN, AIN, Bi₂ Te₃, ZnO, and others can be used.

Materials Characterization. One method of characterization includes a tapping mode. AFM measurements were made using a Digital Instruments Dimension 3100 microscope in combination with a Nanoscope IV Controller (Veeco Metrology, Santa Barbara, Calif.). For example, all measurements were obtained using uncoated, n-doped Si SPM probes (e.g., cantilever length, about 125 μm; resonant frequency, about 300 kHz; spring constant, about 40 N/m; model MPP-11100, Nanodevices, Inc., Santa Barbara, Calif.). In some cases, metallized protein structures were severed using a focused ion beam (FIB; FEI-Strata DB235, Hillsboro, Oreg.) operated using a beam current of about 100 pA. SEM data was obtained from a LEO 1530 scanning electron microscope operating at an accelerating voltage of about 3 keV with an about 8-mm working distance and using magnifications of about 1700× to about 25,000×. In some cases, images were captured using an in-lens annular detector. Current-voltage data was collected using a Karl Suss PM5 probe station coupled to an Agilent 4145B semiconductor parameter analyzer. Tungsten filaments (e.g., about 2-μm radius) were used to probe the structures. In some studies, conductivity measurements were acquired using a CH Instruments 440 potentiostat (Austin, Tex.) interfaced to a PC. Transmission images of intact and severed protein wires were obtained using a Photometrics CoolSnap HQ CCD digital camera (Tuscon, Ariz.) mounted to the Axiovert fabrication microscope and interfaced to Metamorph imaging software (Universal Imaging Corporation, version 6.2, Downingtown, Pa.). Confocal images were acquired using a Leica SP2 AOBS confocal microscope outfitted with a 40× plan-apo 1.25 numerical aperture UV objective; biotin-fluorescein fluorescence was imaged on this system using the 488-nm line from an argon-ion laser and a FITC filter set.

Results and Discussion. Generally, crosslinking of protein-residue side-chains in the present invention can be promoted by type I (direct radical) and type II (oxygen-dependent) photosensitizers (9-11), and has been controlled using near-infrared multiphoton excitation (MPE) to create rugged, surface-adherent matrices that, in some cases, retain the functionality of their protein constituents (12-14). The present invention includes high-intensity laser light focused to submicrometer dimensions by a high numerical aperture microscope objective; the nonlinear dependence of photosensitizer excitation on laser intensity restricts the reaction both radially (i.e., in the focal plane) and axially (i.e. along the optical axis), resulting in a protein crosslinking volume element (referred to as “voxel”) that can be less than 1 fL (15). By translating the relative position of the voxel across a coverslip immersed in a solution of protein and photosensitizer, a continuous matrix can be fabricated with feature sizes as small as about 250 nm. The present invention includes a laser to crosslink proteins. The skilled artisan will recognize that many different types of laser may be used. The present invention includes a laser beam (e.g., a Ti:S laser at about 740 nm) was used to excite FAD and methylene blue, molecules that were used to efficiently sensitize the crosslinking of various proteins, including avidin, BSA, and the heme proteins, cytochrome c and myoglobin. In addition, the heme protein, cytochrome c, can efficiently photosensitize its own crosslinking. Electrical conductivity measurements were obtained ex situ (i.e., on dried, metallized photocrosslinked cytochrome c matrices). The present invention targets metal nanoparticle delivery to photofabricated protein matrices using protein-protein interactions. In one example, gold nanoparticles are coated with a protein that has an isoelectric point (pI) significantly different from that of the matrix protein, with the solution buffered at a pH intermediate to the two pls. In the moderately basic solutions provided as supports for protein-coated nanoparticles (pH 8.8-9.0), planar structures fabricated from cytochrome c (e.g., pI=9.4; Ref. 16) showed a high capacity for binding nanoparticles coated with b-BSA, a strongly acidic protein with a native isoelectric point of 4.8 (17).

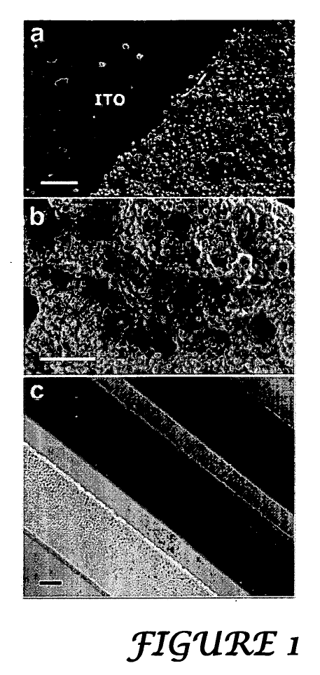

With reference to FIGS. 1a-1c are scanning electron micrograph images of high-density metallization of matrices comprised of photocrosslinked cytochrome c (cyto c). FIG. 1(a) Scanning electron micrograph (SEM) image depicting the interface between a cytochrome c structure (following nanoparticle binding and growth) and an ITO-coated glass substrate; scale bar, 0.5 μm. FIG. 1(b) is a high magnification scanning electron micrograph image demonstrating the tight clustering of reductively grown gold nanoparticles supported on a porous cytochrome c scaffold; scale bar, 0.5 μm. FIG. 1(c) is a scanning electron micrograph image of crosslinked bovine serum albumin (BSA; middle lane), cytochrome c (lower left), and cytochrome c blocked with BSA (upper right) following application and reductive growth of gold nanoparticles. Structures were fabricated on an ITO substrate; non-conductive regions appear dark in this image. Scale bar, 5 μm. For all structures fabricated in FIGS. 1(a), 1(b) and 1(c), cytochrome c was photocrosslinked in a solution containing about 100 mg/mL cytochrome c, about 18 mM phosphate buffer, about 0.1 M sodium perchlorate, and about 4.5 mM FAD; BSA structures were prepared in about 200 mg/mL BSA, about 20 mM HEPES, about 0.1 M NaCl, and about 0.6 mM methylene blue.

Modification of primary amine sites during crosslinking may lower isoelectric points for biotinylated proteins. As can be seen from these images, particles were bound at densities sufficient to form a fully covered surface after reductive growth had enlarged particles to about 50 nm. Pre-treatment of b-BSA nanoparticles with solution-phase avidin could be used to block association of nanoparticles with cytochrome c structures (data not shown). Similarly high levels of b-BSA nanoparticle loading were achieved for structures comprised of another basic protein, avidin (e.g., pI>10; Ref. 18), and nanoparticles coated with HRP and b-HRP (e.g., the C isoform, which has a native isoelectric point of about 8.5 to about 9.0; Ref. 19) were bound by cytochrome c structures at comparable levels.

Consistent with an electrostatic role in binding, structures fabricated from both myoglobin (e.g., pI about 7; Ref. 20) and BSA did not bind appreciable amounts of b-BSA nanoparticles. Moreover, treatment of cytochrome c matrices with solution-phase BSA (e.g., about 200 mg/mL in a HEPES/methylene blue solution for about 5 to about 10 min) before addition of b-BSA nanoparticles efficiently blocked nanoparticle association.

FIG. 1c demonstrates selective metallization of protein matrices based on these results. The usefulness of biopolymers as scaffolds for electronic and electrochemical materials depends critically on the ability to accurately construct complex arrangements of components. The multiphoton photodeposition of the present invention are supporting protein matrices fabricated with well-defined morphologies in three dimensions, and with minimum feature sizes that approach those reported for randomly placed biopolymer-templated wires.

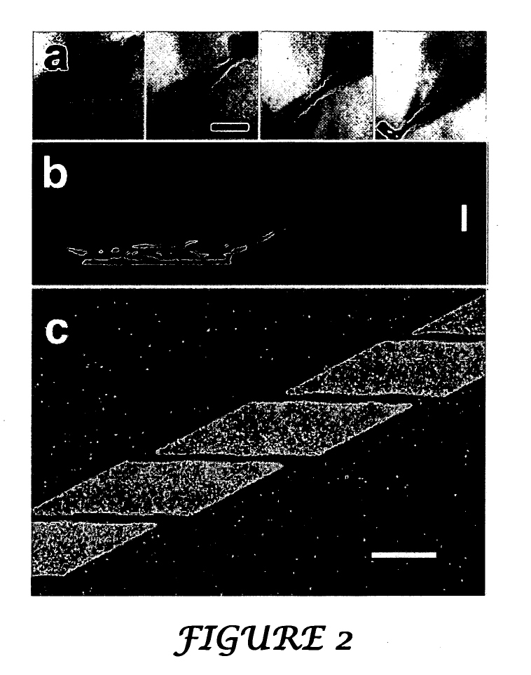

FIGS. 2a-2c are transmission images illustrating detailed control of metallized-protein architectures in two and three dimensions. FIG. 2(a) is a sequence of transmission images showing an avidin cable, tethered only at its ends, that was fabricated diagonally through solution between two spaced glass coverslips. In the left panel, the lower surface of the upper coverslip is in focus; the subsequent panels focus downward in steps of 24 μm, 24 μm and 28 μm, with the right panel showing a portion of the lower tethering region. The cable appears dark as a result of gold nanoparticle binding and growth. Scale bar, 40 μm. FIG. 2(b) is a confocal reconstruction made from an image stack depicting the “side view” of a second avidin cable. The sample was labeled with fluoresceinbiotin and gold nanoparticles, but was not subjected to further growth of nanoparticles. The top tethering region of this cable extended just beyond the depth of focus. Scale bar, 20 μm. FIG. 2(c) is a scanning electron micrograph image of a series of metallized cytochrome c parallelograms fabricated on an ITO coverslip. Scale bar, 10 μm.

Again referring to FIGS. 2a and 2b, images that demonstrate capabilities for fabricating and metallizing crosslinked-protein cables that extend through solution, unsupported, for nearly 100 μm between two opposing coverslips. These large-aspect-ratio diagonal structures were fabricated from avidin by scanning the stage laterally at several microns per second while simultaneously translating the depth of the focal point within the sample solution. The present invention also includes a variety of other geometries using crosslinked proteins, including horizontal cables that extend between co-planar shelfs and arcs that loop from a single surface. FIG. 2c shows one example of two-dimensional patterning (e.g., a series of metallized parallelograms resembling a braided rope). Specific avidin-biotin recognition may assist electrostatic binding in the association of biotinylated-protein particles with avidin matrices.

ITO substrates were patterned with about 50 to 100 μm insulating breaks of bare glass to determine the metallized protein matrices ability to electronically conduct. The cytochrome c structures of the present invention were fabricated across the electronic barriers with overlapping at their ends with the conductive ITO surfaces. In some examples, significant differences were found in photocrosslinking cytochrome c on the glass and ITO surfaces: the fabrication process on glass is less controllable, typically requiring greater laser powers and higher concentrations of cytochrome c and resulting in less uniform matrices that are more highly porous than those patterned on ITO. In addition, AFM topographical analysis indicated that the height of metallized cytochrome c matrices constructed across insulating glass regions generally ranged from about 1.5 to 3.0 μm, as compared to about 700 nm on ITO surfaces.

The decreased ability of the glass substrate to dissipate local heating (dependent on thermal conductivity) plays an important role in determining matrix structure, as diffusion and convection result in more rapid depletion of reactive photoproducts from the multiphoton focal volume. Notably, matrices including various other proteins, including avidin, could be fabricated more controllably on glass substrates than structures formed from cytochrome c. Although avidin matrices efficiently bind gold nanoparticles, they were not used in initial conductivity studies because of higher non-specific adsorption of avidin to glass and, hence, greater background binding of nanoparticles.

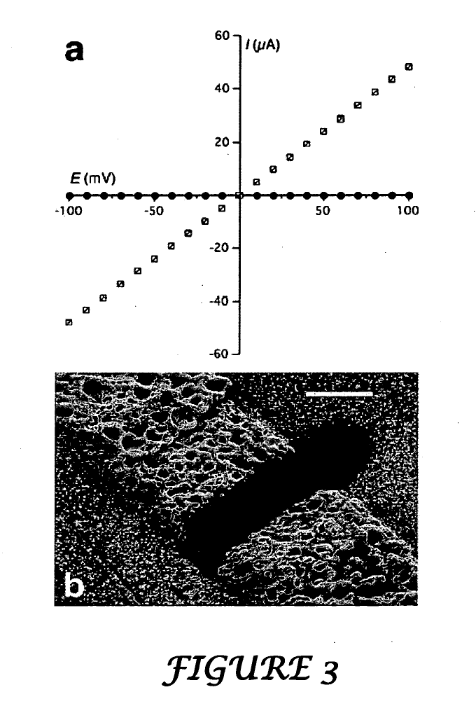

FIG. 3(a) is a graph of the conductivity measurements of metallized cyt c matrices. FIG. 3(a) Current potential (I-V) measurements on a representative sample in which a metallized cyt c matrix spanned an insulating gap (e.g., about 68 μm) between ITO electrodes (squares) and after the matrix was severed (darkened circles). Ohmic scaling (I-V) measurements performed on a representative metallized cytochrome c structure are shown in FIG. 3a. Tungsten probes were placed in contact with ITO adjacent to the ends of the protein wires. Conductivities for structures fabricated across insulating gaps were determined to range from about 6 to about 14Ω⁻¹ cm¹. Importantly, conductivities were nearly zero unless nanoparticles were both applied to structures and subjected to reductive growth. The wires of the present invention may be severed using FIB milling.

FIG. 3(b) SEM depicting the metallized cytochrome c matrix after severing with a focused ion beam (FIB). Although some non-specific deposition of gold can be seen in the vicinity of the structure, FIB disruption of the matrix decreased conductivity by more than 106-fold. Scale bar, 5 μm. Solutions used to fabricate protein matrices for conductivity measurements contained 200 mg/mL cytochrome c with no additional photosensitizer. FIG. 3(b) shows a several-micron-long cut made through the middle section of a wire, a disruption that virtually eliminated current flow (e.g., at 100 mV the severed structure supported a current of 2 pA versus 50 μA in the intact structure). As further confirmation that current responses could not be attributed to nonspecific adsorption of protein (and subsequent gold deposition) on the glass surface, control studies in which patterned ITO slides were subjected to identical solutions and procedures as test slides, with the exception that protein photocrosslinking was not performed. Currents measured for these controls were about 1 pA at an applied potential of about 100 mV, and did not clearly scale with potential.

In these initial measurements of metallized cytochrome c conductivity, contact resistance between the protein matrix and the ITO surfaces appears to be a limiting factor. To evaluate the magnitude of the effect, several additional samples (with matrix diameters ranging from about 1.5 μm to about 10 μm) were characterized by placing the tungsten probes in direct contact with metallized cytochrome c structures. Although probe contact caused some damage to protein matrices, measured conductivities increased to about 103Ω⁻¹ cm⁻¹, nearly 100-fold greater than determined with the probes placed on the ITO surfaces. Another source of error in calculating conductivities is the simplifying assumption that matrices are solid (i.e., contain no void volume), a poor approximation given the high level of porosity of the cytochrome c structures fabricated for conductivity measurements (e.g., FIG. 3b). The present invention demonstrates that controlled fabrication of highly conductive gold nanoparticle-protein composites is possible through a direct-write, photodeposition procedure.

Unlike other approaches for templating electronic materials using biological molecules, the present invention can be used to deposit scaffolds with precise spatial control in three dimensions. As the metallized protein matrices of the present invention can be fabricated with a broad range of geometries and opens significant opportunities for creating optical, structural and electronic components (e.g., electrochemical and plasmon-based sensors, inductive heating elements) in chemically sensitive and mechanically confined environments. The present invention allows the formation of functional bioelectronic architectures for monitoring and stimulating biological processes, e.g., nanowire.

Innervating the matrix with photocrosslinked protein wires. The present invention includes a method for fabricating electronic materials using biomolecular scaffolds that can be constructed with precisely defined three-dimensional topographies having feature sizes that range from about 200 nm to several millimeters. In one example, structures are created using a tightly focused pulsed laser beam capable of promoting protein photocrosslinking in specified femtoliter volume elements is scanned within a protein solution, creating biomolecular matrices that either remain in integral contact with a support surface or extend as freestanding structures through solution, tethered at their ends and interconnected with other electronic components. Once fabricated, specific protein scaffolds can be selectively metallized (or developed with metal oxides) via targeted deposition and growth of nanoparticles, yielding high-quality bioelectronic materials.

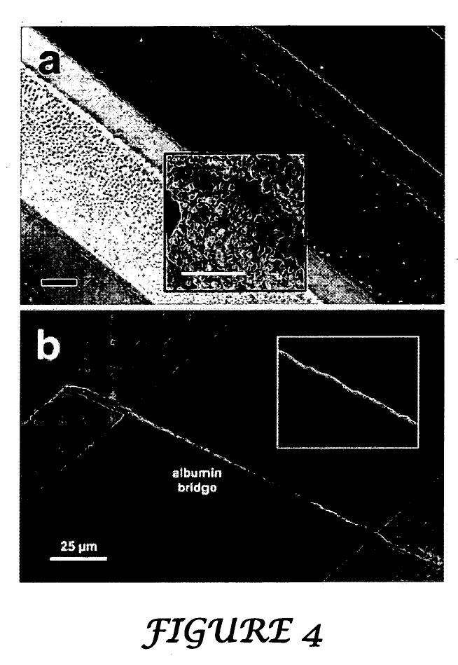

Now referring to FIGS. 4a and 4b are high-density metallization of matrices comprised of photocrosslinked cytochrome c. FIG. 4a is a scanning electron micrograph (SEM) of crosslinked bovine serum albumin (BSA; middle lane), cytochrome c (lower left), and cytochrome c blocked with BSA (upper right) following application and reductive growth of gold nanoparticles. Structures were fabricated on an ITO substrate; non-conductive regions appear dark in this image. Scale bar, 5 μm. Inset is a scanning electron micrograph image showing tight clustering of gold nanoparticles supported on a porous cytochrome c scaffold following binding and growth; scale bar, 0.5 μm. FIG. 4b is a scanning electron micrograph image of the BSA bridge fabricated across a gap between glass coverslips that extends for >100 μm. Inset is a Close-up image of the BSA cable.

Interrogating biowire electrodes for efferent and afferent connections. The ingrowth of axons is largely random and the regenerated neurites would be a mix of sensory and motor axons. Hence, it is necessary to determine the type and function of each axon that is associated with the electrodes. The first step is to determine which electrodes receive signals from regenerated motor axons by recording from all electrodes while the host is attempting movement. Those electrodes that detect signals are considered to be associated with a motor axon; those that are silent are considered candidates for being associated with a sensory axon. To determine whether these silent electrodes are associated with sensory axons each would be interrogated with a stimulus pulse.

To deal with the issue that the extremely high density of photocrosslinked protein wires envisioned for this device may exceed the “pin out” capacity, the device may be fitted with a microelectronic headstage capable of rapidly switching between several electrodes so that one lead functions to connect to several electrodes.

It will be understood that particular embodiments described herein are shown by way of illustration and not as limitations of the invention. The principal features of this invention can be employed in various embodiments without departing from the scope of the invention. Those skilled in the art will recognize, or be able to ascertain using no more than routine experimentation, numerous equivalents to the specific procedures described herein. Such equivalents are considered to be within the scope of this invention and are covered by the claims.

All of the compositions and/or methods disclosed and claimed herein can be made and executed without undue experimentation in light of the present disclosure. While the compositions and methods of this invention have been described in terms of preferred embodiments, it will be apparent to those of skill in the art that variations can be applied to the compositions and/or methods and in the steps or in the sequence of steps of the method described herein without departing from the concept, spirit and scope of the invention. All such similar substitutes and modifications apparent to those skilled in the art are deemed to be within the spirit, scope and concept of the invention as defined by the appended claims.

### REFERENCES

(1) Seeman, N. C. & Belcher, A. B. (2002) Proc. Natl. Acad. Sci. USA 99, 6451-6455.
(2) Aizenberg, J., Muller, D. A., Grazul, J. L., & Hamann, D. R. (2003) Science 299, 1205-1208.
(3) Monson, C. F. & Woolley, A. T. (2003) Nano Lett. 3, 359-363.
(4) Braun, E., Eichen, Y., Sivan, U., & Ben-Yoseph, G. (1998) Nature 391, 775-778.
(5) Keren, K., Krueger, M., Gilad, R., Ben-Yoseph, G., Sivan, U., & Braun, E. (2002) Science 297, 72-75.
(6) Scheibel, T., Raghuveer, P., Sawicki, G., Lin, X.-M., Jaeger, H., & Lindquist, S. L. (2003) Proc. Natl. Acad. Sci. USA 100, 4527-4532.
(7) Reches, M. & Gazit, E. (2003) Science 300, 625-627.
(8) Patolsky, F., Weizmann, Y., Willner, I. (2004) Nature Materials 3, 692-695.
(9) Spikes, J. D., Shen, H.-R., Kopeceková, P. & Kopecek, J. (1999) Photochem. Photobiol. 70, 130-137.
(10) Verweij, H. & Van Steveninck, J. (1982) Photochem. Photobiol. 35, 265-267.
(11) Shen, H.-R., Spikes, J. D., Kopeceková, P. & Kopecek, J. (1996) J. Photochem. Photobiol. B 34, 203-210.
(12) Pitts, J. D., Howell, A. R., Taboada, R., Banerjee, I., Wang, J., Goodman, S. L. & Campagnola, J. (2002) Photochem. Photobiol. 76, 135-144.
(13) Pitts, J. D., Campagnola, P. J., Epling, G. A. & Goodman, S. L. (2000) Macromolecules 33, 1514-1523.
(14) Kaehr, B., Allen, R., Javier, D. J., Currie, J., Shear, J. B. (2004) Proc. Natl. Acad. Sci. USA 101, 16104-16108.
(15) Shear, J. (1999) Anal. Chem. 71, 598A-605A.
(16) Righetti, P. G. & Caravaggio, T. (1976) J. Chromatogr. 127, 1-28.
(17) Peters, T. (1985) Adv. Prot. Chem. 37, 161-245.
(18) Melamed, M. D. & Green, N. M. (1963) Biochem. J. 89, 591-599.
(19) Yamazaki, I. and Nakajima, R. (1986) in Molecular and physiological aspects of plant peroxidases, eds. Greppin, H., Penel, C., Gaspar, T. (University of Geneva Press, Geneva, Switzerland), pp. 71-84.
(20) Malamud, D. & Drysdale, J. W. (1978) Anal. Biochem. 86, 620-647.
(21) Basu, S.; Campagnola, P. J. “Ezymatic Activity of Alkaline Phosphatase Inside Protein and Polymer Structures Fabricated via Multiphoton Excitation,” Biomacromol. 2004, 5, 572-579.
(22) Kaehr, B.; Allen, R.; Javier, D. J.; Currie, J.; Shear, J. B. “Guiding Neuronal Development with in situ Microfabrication,” PNAS 2004, 101 16104-16108; Hill, R. T.; Lyon, J. L.; Allen, R.; Stevenson, K. J.; Shear, J. B. “Aqueous Microfabrication of Bioelectronic Architectures,” PNAS (submitted).

1. A method of making a metallized biomolecular scaffold comprising the steps of:
crosslinking a polymer matrix with a photon beam to form a crosslinked matrix, wherein the crosslinked matrix comprises one or more polymers crosslinked to one or more crosslinking agents; and
binding one or more metal nanoparticles with the crosslinked matrix.

2. The method of claim 1, wherein the one or more metal nanoparticles is coasted with one or more proteins.

3. The method of claim 1, wherein the one or more polymers comprises cytochrome c.

4. The method of claim 1, wherein the one or more polymers are made from monomers comprising one or more photopolymerizable organic monomers.

5. The method of claim 1, wherein the one or more crosslinking agents comprises cytochrome c.

6. The method of claim 1, wherein each photon of the photon beam has a wavelength in at least one of the deep red, red, infrared, visible and ultraviolet segments of the electromagnetic spectrum.

7. The method of claim 1, wherein the photon beam is produced by one or more Titanium sapphire lasers.

8. The method of claim 1, wherein the metallized biomolecular scaffold is in integral contact with a support surface, extends as freestanding structures through a solution or a combination thereof.

9. The method of claim 1, wherein the metallized biomolecular scaffold is conductive.

10. The method of claim 1, wherein the metal nanoparticles comprises one or more pure metals, one or more semiconductor, one or more metal oxides and combinations and mixtures thereof.

11. A system for forming a nanoscale structure in a solution comprising:
a chamber suitable for nanoparticle metallization of a polymer positioned to receive one or more photons from an optical system comprising an imaging mechanism interfaced with a multiphoton excitation laser, wherein the one or more photons crosslink the one or more polymers and one or more photosensitizers in the chamber prior to the nanoparticle metallization.

12. The system of claim 11, further comprising one or more detectors positioned relative to the chamber to record spectroscopic characteristics, optical characteristics, electrochemistry characteristics or a combination thereof.

13. An electrical conductive nanoscale architectural matrix comprising:
one or more metal nanoparticles bound to an architectural matrix comprising a multi-photon beam induced crosslink between one or more polymers and one or more photosensitizers.

14. The device of claim 13, one or more polymers are made from monomers comprising one or more photopolymerizable organic monomers, photopolymerizable inorganic monomers, cross-linkers, monomers having at least one olefinic bond, oligomers having at least one olefinic bond, polymers having at least one olefinic bond, olefins, halogenated olefins, acrylates, methacrylates, acrylamides, bisacrylamides, styrenes, epoxides, cyclohexeneoxide, amino acids, peptides, proteins, fatty acids, lipids, nucleotides, oligonucleotides, synthetic nucleotide analogues, nucleic acids, sugars, carbohydrates, cytokines and combinations or mixtures thereof.

15. The device of claim 13, wherein the one or more polymers comprises cytochrome c, cytochrome c oxidase, cytochrome c peroxidase, horseradish peroxidase, fibrinogen, trimethylolpropane triacrylate, avidin, bovine serum albumin, and the heme proteins, myoglobin or combinations and mixtures thereof.

16. The device of claim 13, wherein the one or more photosensitizers comprise flavin adenine dinucleotide, heme proteins, cytochrome c, methylene blue or combinations and mixtures thereof.

17. The device of claim 13, wherein the nanoscale architectural matrix is in integral contact with a support surface, extends as freestanding structures through a solution or a combination thereof.

18. The device of claim 13, wherein the one or more nanoparticles comprise one or more bound proteins.

19. The device of claim 13, wherein the metal nanoparticles comprises pure metals, semiconductor, metal oxides and combinations and mixtures thereof.

20. The metallized nanostructure made by the method of claim 1.

21. The method of claim 1, wherein the one or more metal nanoparticles is coated with one or more agents that promote binding.

22. The method of claim 1, further comprising one or more agents coated on at least a portion of the one or more metal nanoparticles to promote binding or one or more compositions to the one or more metal nanoparticles.
